(19) United States
(12) Patent Application Publication
(10) Pub. No.: US 2021/0340611 A1
(43) Pub. Date: Nov. 4, 2021

(54) DIGITAL POLYMERASE CHAIN REACTION METHOD FOR DETECTING NUCLEIC ACIDS IN SAMPLES

(71) Applicant: ACADEMIA SINICA, Taipei City (TW)

(72) Inventor: Kuo-Ping CHIU, Taipei City (TW)

(73) Assignee: ACADEMIA SINICA, Taipei City (TW)

(21) Appl. No.: 17/283,809

(22) PCT Filed: Oct. 9, 2019
(86) PCT No.: PCT/US2019/055342

§ 371 Date: Apr. 8, 2021

Related U.S. Application Data
(60) Provisional application No. 62/743,149, filed on Oct. 9, 2018.

(51) Int. Cl. C12Q 1/6855

(52) U.S. Cl. CPC C12Q 1/6855

(57) ABSTRACT

The present invention relates to a method for detecting nucleic acid (NA) molecules in samples. More particularly, the present invention relates to an improved digital PCR-based method for detecting specific nucleic acid sequence(s). The present invention is useful for research and diagnostic applications with increased sensitivity and accuracy. The present invention also provides a kit for performing the method for assessingnucleic acids in samples as described herein.

### RELATED APPLICATIONS

This application claims the benefit of U.S. provisional application No. 62/743,149, filed Oct. 9, 2018 under 35 U.S.C. § 119, the entire content of which is incorporated herein by reference.

### TECHNOLOGY FIELD

The present invention relates to a method for detecting nucleic acid (NA) molecules in samples. More particularly, the present invention relates to an improved digital PCR-based method for analyzing nucleic acid fragments. The present invention is useful for research and diagnostic applications with increased sensitivity and accuracy. The present invention also provides a kit for performing the method for detecting nucleic acids in samples as described herein.

### BACKGROUND

Various techniques for nucleic acid detection have been developed for many researches and diagnostic applications. Digital polymerase chain reaction (dPCR) is considered one of the most sophisticated quantitative method for analyses of gene copy number variations (CNVs), gene expression, genetic mutations and single-nucleotide polymorphisms (SNPs). Its popularity keeps on increasing. A number of companies have designed company-specific experimental approaches, hardware and software applications (Dong et al., 2015; Morley, 2014; Zhao et al., 2016).

The most prevalent models are droplet-based and titer plate-based digital PCR methods. Current droplet-based digital PCR is commercialized by Bio-Rad. QX200 Droplet Digital PCR (ddPCR) machine, as recently commercialized by Bio-Rad, is one of the most advanced model that combines microfluidics and surfactant chemistries to divide PCR reactions into water-in-oil droplets for absolute nucleic acid quantification (Hindson et al., 2011). This approach allows near 20,000 nanoliter-sized droplets to be analyzed per sample per run, making it the most efficient approach among the instruments of the same kind. On the other hand, titer plate-based digital PCR, as exemplified by Qiagen (http://www.captodayonline.com/high-throughput-digital-per-system-1017/), runs reactions in titer plates. For this approach, DNA molecules are diluted and partitioned into, e.g., 96-well titer plates, for independent PCR reactions. After amplification, all wells are probed and quantified by gene-specific sequences to identify wells with positive reactions.

Digital PCR, as a PCR-based technology for nucleic acid detection, normally requires paired primers for amplification and probe(s) for detecting target nucleic acids, where samples are diluted and partitioned so that nucleic acid fragments therein are separated for independent reactions, each of which with very limited number of the target nucleic acid molecules. In this way, the PCR reaction can be individually carried out within each partition and the signal in each partition is determined as negative or positive and therefore the exact amount of copies of a nucleic acid sequence in the original sample can then be determined by counting the number of positive partitions (sequence detected) versus negative partitions (sequence not detected) based on Poisson distribution. In general, the experimental procedure of digital PCR comprises a few steps: 1) dilution of target DNA molecules; 2) partition of well-separated target DNA fragments into discrete droplets/chambers, each of which also contains other required components for amplification and signal detection; 3) PCR amplification using “paired” gene-specific primers targeting “internal” regions of gene(s) of interest; 4) detection of fluorescent signals using fluorescent probe(s); 5) quantification of positive and negative reactions based on Poisson distribution; and 6) cross-sample comparison to determine the significance.

Cell-free nucleic acid samples are easy-accessible, noninvasive genetic materials with increasing popularity for the diagnosis of a number of diseases (Wagner, 2012). These genetic materials are released from all cells in the body, including normal cells, diseased cells and microbes. In theory, cell-free nucleic acids may present in many types of body fluids including blood, saliva, urine, vaginal discharge, seminal fluid, lymph and sweat (Chiu and Yu, 2019; Nai et al., 2017; Wagner, 2012). Although with numerous advantages, cell-free nucleic acid samples are frequently of low quantity, making it difficult to manipulate and thus easy to get lost during experimental process. Moreover, cell-free nucleic acids are also highly fragmented. These represent a serious problem especially for such precious materials.

Current digital PCR still confronts many challenges for analysis of circulating cell-free nucleic acids. Some approaches are suggested to improve current digital PCR, such as proper preservation of nucleic acid samples to reduce the degradation of cfDNAs, efficient purification e.g. silica membrane to collect sufficient amounts of cfDNAs to be detected, and high-sensitivity capillary electrophoresis to enhance the sensitivity of detection. Current digital PCR also has drawbacks that probes are designed based on specific known mutation sites to detect certain mutation in nucleic acids which however do not cover all genetic variations and thus restrict the testing of other potential variations or mutations.

Thus, there is a need to develop an improved digital PCR method for nucleic acid detection.

### SUMMARY

The present invention provides an improved digital PCR for nucleic acid detection in a sample, called digital T-oligo primed PCR (digital TOP-PCR). The digital TOP-PCR of the present invention features use of a homogeneous adapter (HA) ligated to the termini of all nucleic acid fragments in the sample and a single-type primer (T/U oligo) that recognizes a complementary sequence in HA and acts as both forward and reverse primers to perform amplification. The digital TOP-PCR of the present invention allows amplification of all nucleic acid fragments in the sample such that subsequent detection of target nucleic acids can be achieved with increased sensitivity, especially reducing the false negative rate.

In particular, the present invention provides a method for analyzing nucleic acids in a sample, said sample containing one or more linear, double stranded nucleic acid fragment(s) (NA fragment(s)), said method comprising the steps of:

(a) subjecting the sample to a 3′-A tailing reaction allowing for adding an adenine nucleotide (A) to the 3′-tail of the NA fragment(s) to produce 3′-adenine nucleotide (3′-A) overhang nucleic acid fragment(s) (3′-A overhang NA fragment(s));

(b) providing a 3′-thymine (T) or 3′-uracil (U) nucleotide overhang double-stranded homogenous adapter which comprises a P oligo strand carrying a 5′-phosphate for ligation to the 3′-A overhang NA fragment(s) and a T/U oligo strand carrying a 3′-T or 3′-U without a 5′-phosphate, wherein the T/U oligo strand is complimentary to the P oligo strand except at the 3′-T or 3′-U of the T/U oligo strand;

(c) subjecting the sample of (a) to a ligation reaction allowing for ligating the homogenous adapter to the 3′-A overhang NA fragment(s) at both ends to produce adapter-ligated nucleic acid fragment(s) (adapter-ligated NA fragment(s));

(d) combining the sample of (c) with polymerase chain reaction (PCR) reagents and detection reagents to provide amplification/detection-ready sample, wherein the PCR reagents include a single-type primer having the nucleic acid sequence of the T/U oligo strand for amplification and the detection reagents include one or more fluorescent probe(s) producing fluorescent signals and specifically hybridizing with the NA fragment(s) for detection;

(e) partitioning the amplification/detection-ready sample of (d) into a plurality of partitions, each containing limited copy of the adapter-ligated NA fragment(s);

(f) performing PCR in each partition using the adapter-ligated NA fragment(s) as template(s) and the single-type primer as both forward and reverse primers to amplify the adapter-ligated NA fragment(s); and

(g) assessing the fluorescent signal(s) in each fraction.

In some embodiments, step (g) includes determining a droplet/fraction to be positive or negative based on the intensity of a fluorescent signal and later to calculate the total number (counts) of droplets/fractions with a positive signal.

In some embodiments, in step (e), more than 50% of the partitions contain no more than one copy of the adapter-ligated NA fragment(s).

In some embodiments, in step (e), each partition contains at least one copy of the adapter-ligated NA fragment(s).

In some embodiments, the NA fragment(s) comprises a nucleic acid sequence that is indicative of a healthy/diseased state of the subject.

In some embodiments, the homogenous adapter of step (b) does not self-ligate.

In some embodiments, the homogenous adapter of step (b) has 3′-T or 3′U overhang in its T/U oligo strand and 3′-non-A overhang in its P oligo strand.

In some embodiments, the homogenous adapter of step (b) has one end which is 3′-T overhang and the other end which is a blunt end.

In some embodiments, the sample is obtained from a body fluid, including, but not limited to, blood, urine, saliva, tears, sweat, breast milk, nasal secretions, amniotic fluid, semen, and vaginal fluid.

In some embodiments, the NA fragment(s) in the sample are cell-free DNAs (cfDNAs).

In some embodiments, prior to step (a) the method as descried herein further comprises performing an end-repair reaction to the NA fragments.

In some embodiments, the PCR of step (f) is performed by oil emulsion or droplet PCR, or well-based PCR.

In some embodiments, the assaying of step (g) is performed by flow cytometry using fluorescent probes.

In some embodiments, the method of the present invention comprise the steps of (a) to (g) and optional step (a)′: if the NA fragment(s) include liner, single-stranded RNAs, subjecting the sample prior to step (a) to a reverse transcription-PCR (RT-PCR) to convert the RNAs to linear, double-stranded complementary DNA (cDNA).

The present invention also provides a kit for performing the method for detecting nucleic acid fragment(s) in samples as described herein. Specifically, the kit comprises

(i) adapter ligation reagents comprising a homogenous adapter, a ligation buffer and a ligase, wherein the homogenous adapter comprises a P oligo strand carrying a 5′-phosphate and a T/U oligo strand carrying a 3′-T or 3-U without a 5′-phosphate, wherein the T/U oligo strand is complimentary to the P oligo strand except at the 3′-T or 3′-U of the T/U oligo strand, wherein the homogenous adapter is capable of ligating to the nucleic acid fragment(s) at both ends, wherein the nucleic acid fragment(s) has a 3′-A overhang;

(ii) PCR reagents comprising a single-type primer (the sole primer) having the nucleic acid sequence of the T/U oligo strand, dNTPs, a PCR buffer, and a DNA polymerase; and

(iii) detection reagents comprising one or more detectable probes having complementary sequences specifically hybridizing with the nucleic acid fragment(s).

In some embodiments, the kit further comprises instructions for use, wherein the instructions for use comprise instructions for performing the method comprising steps (a) to (g) as described herein.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1 shows that the procedure of the method of the present invention. P oligo: 5′-GTCGGAGTCTgcgc-3′ (SEQ ID NO: 24). T-oligo: 5′-AGACTCCGAC(T)-3′ (SEQ ID NO: 23).

FIG. 2 shows the differences between the method of the present invention and the conventional PCR-based detection method. A cfDNA sample contains a pool of cfDNA fragments with random breakages from genomic origins. In conventional PCR-based detection method, only cfDNA fragment “g” covering both the first given primer binding site and the second given primer binding site can be amplified and detected; as a result, the sensitivity of detection is limited and particularly when the nucleic acid content is low, the sensitivity can be even worse. On the contrary, in the method of the present invention, all cfDNA fragments can be evenly and universally amplified and besides after such amplification not only fragment “g” but also other fragments “a” to “f” (with only one primer binding site or even without any of the primer binding sites), all originating from the same target pathogenic nucleic acid (the same genomic origin), can be detected, for example, using one or more probe(s) with detectable label(s) capable of specifically hybridizing with any region within the target pathogenic nucleic acid; as a result, the sensitivity of detection is increased and false negatives can be minimized since the amplification before detection is equally (non-specifically) applicable to all nucleic acid fragments and thus the relative amount of each of the amplified nucleic acid fragments can represent to that present in the original sample.

FIG. 3 shows the counts of positive droplets produced from conventional ddPCR and ddTOP-PCR methods using samples containing variable amounts of partial and full templates. Lanes 1 and 2 (A05 and A06 controls) were generated from 5′ primer binding site-deleted NAGK gene sequence using T/U oligo primer and N-myc gene-specific primers, respectively, for amplification, while the rest (lanes 3-16, or B05-H06) are generated from mixed templates containing variable amounts (100%-0%) of partial (labeled as ‘H’, or 5′ primer binding site-deleted) and full (labeled as ‘F’, or with both primer binding sites) templates. All samples used the same N-myc probe for detection. Counts from ddTOP-PCR are labeled with ‘05’ behind letter, while their corresponding counterparts of ddPCR are labeled side-by-side with ‘06’. Digits in the figure represent counts (copies/microliter). A total of 20 microliter for each sample was used for counting by QX200 ddPCR machine. Ch1, channel 1 as defined by QX200.

FIG. 4 shows the comparison of fluorescent signal intensity between ddPCR and ddTOP-PCR. Same as FIG. 3, lanes 1 and 2 (A05 and A06 controls) were generated from 5′ primer binding site-deleted NAGK template using T/U oligo primer and N-myc primers, respectively, for amplification, while the rest (lanes 3-16, or B05-H06) are generated from mixed templates containing variable amounts (100%-0%) of partial (labeled as ‘H’, 5′ primer binding site-deleted) and full (labeled as ‘F’, with both primer binding sites) templates. All samples used the same N-myc oligo probe for detection. Notice that, droplets shown as black dots were not counted as positives, because their intensities were below default threshold.

FIG. 5 shows the sequences as set forth in Table 1. T oligo: 5′-AGC GCT AGA CTC CGA CT-3′ (SEQ ID NO: 1). P oligo, 5′-GT CGG AGT CTA GCG CT-3′ (SEQ ID NO: 2).

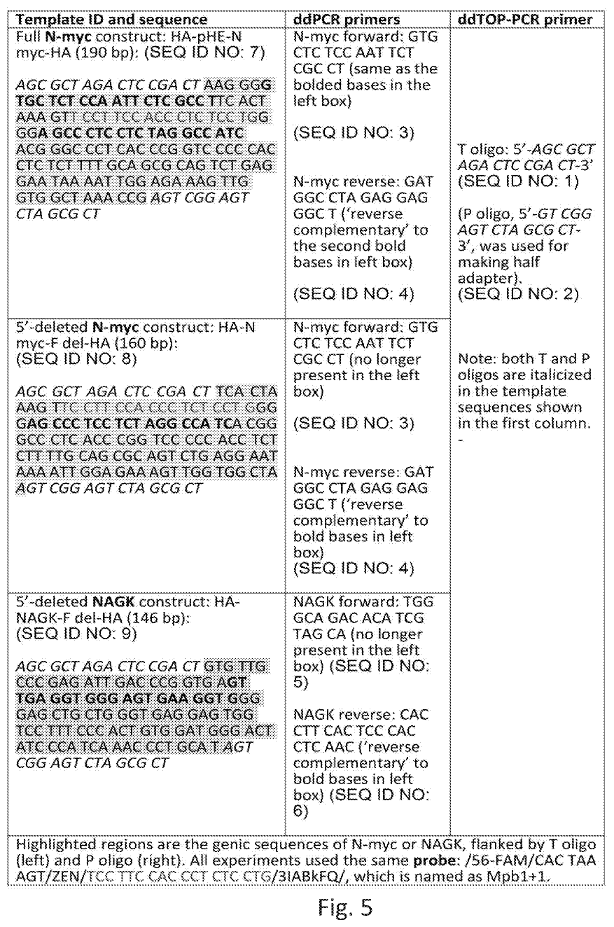

### DETAILED DESCRIPTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “comprise” or “comprising” is generally used in the sense of include/including which means permitting the presence of one or more features, ingredients or components. The term “comprise” or “comprising” encompasses the term “consists” or “consisting of”

As used herein, “around”, “about” or “approximately” can generally mean within 20 percent, particularly within 10 percent, and more particularly within 5 percent of a given value or range. Numerical quantities given herein are approximate, meaning that the term “around”, “about” or “approximately” can be inferred if not expressly indicated.

The term “polynucleotide” or “nucleic acid” refers to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. The term “nucleic acid” typically refers to large polynucleotides. It will be understood that when a nucleotide sequence is represented by a DNA sequence (i.e., A, T, G, C), this also includes an RNA sequence (i.e., A, U, G, C) in which “U” replaces “T.” The term “oligonucleotide” refers to a relatively short nucleic acid fragment, typically less than or equal to 150 nucleotides long e.g., between 5 and 150. Oligonucleotides can be designed and synthesized as needed. In the case of a primer, it is typically between 5 and 50 nucleotides, particularly between 8 and 30 nucleotides in length. In the case of a probe, it is typically between 10 and 100 nucleotides, particularly between 15 and 30 nucleotides in length.

As used herein, the term “complementary” refers to the topological compatibility or matching together of interacting surfaces of two polynucleotides. Thus, the two molecules can be described as complementary, and furthermore the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′.

As used herein, target nucleic acids can refer to particular nucleic acids of interest being detected in a sample. Target nucleic acids include but are not limited to DNA such as genomic DNA, mitochondrial DNA, cDNA and the like, and RNA such as mRNA, miRNA, and the like. Target nucleic acids may derive from any sources including naturally occurring sources or synthetic sources. For example, target nucleic acids may be from animal or pathogen sources including, without limitation, mammals such as humans, and pathogens such as bacteria, viruses and fungi. Target nucleic acids can be obtained from any body fluids or tissues (e.g., blood, urine, skin, hair, stool, and mucus), or an environmental sample (e.g., a water sample or a food sample). In some embodiments, target nucleic acids can be a collection of nucleic acid molecules of the same origin (e.g., from the same gene of normal or diseased subject or pathogens) but in various length. For example, numerous segments of the gene encoding for hepatitis B surface antigen (HBsAg) may be present in a test sample as “target” nucleic acids fragments of various length. Since each of the target nucleic acid molecules contains at least a portion of the HBsAg gene, probes or primers having sequences corresponding (or complementary) to various locations within the HbsAg gene can be used for detection of the target nucleic acid fragments. For another example, target nucleic acids may be those containing genetic mutations (e.g., a single nucleotide polymorphism (SNP) indicative of a disease such as cancer).

As used herein, the term “primer” refers to oligonucleotides that can be used in an amplification method, such as a polymerase chain reaction (PCR), to amplify a target nucleotide sequence. In a conventional PCR, at least one pair of primers including one forward primer and one reverse primer are required to carry out the amplification. Typically, for a target DNA sequence consisting of a (+) strand and a (−) strand to be amplified, a forward primer is an oligonucleotide that can hybridize to the 3′ end of the (−) strand and can thus initiate the polymerization of a new (+) strand under the reaction condition; whereas a reverse primer is an oligonucleotide that can hybridize to the 3′ end of the (+) strand under the reaction condition and can thus initiate the polymerization of a new (−) strand under the reaction condition. Specifically, as an example, a forward primer may have the same sequence as the 5′ end of the (+) strand, and a reverse primer may have the same sequence as the 5′ end of the (−) strand. Normally, a forward primer and a reverse primer useful for amplification of a target nucleic acid sequence are different from each other in sequence. As used herein, a “single” primer refers to only one type of primer, all of which have the same sequence, instead of a pair of primers having distinct sequences, one being a forward primer and the other being a reverse primer.

The term “hybridization” as used herein shall include any process by which a strand of nucleic acid joins with a complementary strand through base pairing. Relevant methods are well known in the art and described in, for example, Sambrook et al., Molecular Cloning: A Laboratory Manual, 2nd ed., Cold Spring Harbor Laboratory Press (1989), and Frederick M. A. et al., Current Protocols in Molecular Biology, John Wiley & Sons, Inc. (2001). Typically, stringent conditions are selected to be about 5 to 30° C. lower than the thermal melting point (Tₘ) for the specified sequence at a defined ionic strength and pH. More typically, stringent conditions are selected to be about 5 to 15° C. lower than the Tₘ for the specified sequence at a defined ionic strength and pH. For example, stringent hybridization conditions will be those in which the salt concentration is less than about 1.0 M sodium (or other salts) ion, typically about 0.01 to about 1 M sodium ion concentration at about pH 7.0 to about pH 8.3 and the temperature is at least about 25° C. for short probes (e.g., 10 to 50 nucleotides) and at least about 55° C. for long probes (e.g., greater than 50 nucleotides). An exemplary non-stringent or low stringency condition for a long probe (e.g., greater than 50 nucleotides) would comprise a buffer of 20 mM Tris, pH 8.5, 50 mM KCl, and 2 mM MgCl₂, and a reaction temperature of 25° C.

As used herein, an “overhang” refers to a stretch of a single unpaired nucleotide or a longer unpaired nucleotides at the end of a linear, double-stranded nucleic acid molecule. The unpaired nucleotide(s) can be in either 3′ or 5′ end, creating either 3′ or 5′ overhangs, respectively. A “3′-A overhang” means the unpaired nucleotide(s) is/are present in 3′-end and it is composed of one or more adenine (A) nucleotides. A “3′-non-A overhang” means the unpaired nucleotide(s) is/are present in 3′-end and it does not include any adenine (A) nucleotide. A “3-T overhang” means the unpaired nucleotide(s) is/are present in 3′-end and it is composed of one or more thymine (T) nucleotide.

A “single,” “homogenous” or “universal” primer means only one type of primer with the same sequence is present, instead of a pair of primers, in the PCR reaction. The term “heterogeneous primers” means at least one paired primers each member having different sequences from each other are present in the PCR reaction.

As used herein, the term “adapter” refers to an oligonucleotide that can be ligated to the ends of a double-stranded nucleic acid molecule. An adapter may be 10 to 50 bases in length, preferably 10 to 30 based in length, more preferably 10 to 20 based in length. Lower than 10 nucleotide in length may decrease specificity for annealing. Higher than 20 nucleotides in length may not be cost-effective. The term a “homogeneous” adapter means one single type of adapter for ligating to both ends of a double-stranded nucleic acid molecule. The term a “heterogeneous” adapter means at least two types of adapters that have different nucleotide sequences from each other, one for ligating to 5′ end and the other for ligating to 3′end of a double-stranded nucleic acid molecule.

To amplify nucleic acid fragments in a sample, we have developed a T-oligo-primed polymerase chain reaction (TOP-PCR) technology by using a homogenous adapter composed of a P-oligo and a T-oligo and ligated to both ends of all the nucleic acid fragments and then applying the T-oligo as a single primer to perform the amplification of all the nucleic acid fragments in the sample without discrimination. See U.S. Pat. No. 10,407,720, the entire content of which is incorporated herein by reference.

In the present invention, it is found that conventional digital PCR can be significantly improved by using a TOP-PCR technology. The method of the present invention is performed for nucleic acid detection in a digital manner by using TOP-PCR for amplification so that all the nucleic acids in the reaction are amplified in equal proportion and target nucleic acids can be detected by one or multiple sequence-specific probes with increased sensitivity. As demonstrated in the examples as provided below, the method of the present invention shows at least about 14% increase in sensitivity compared to conventional digital PCR using a paired PCR primer for amplification.

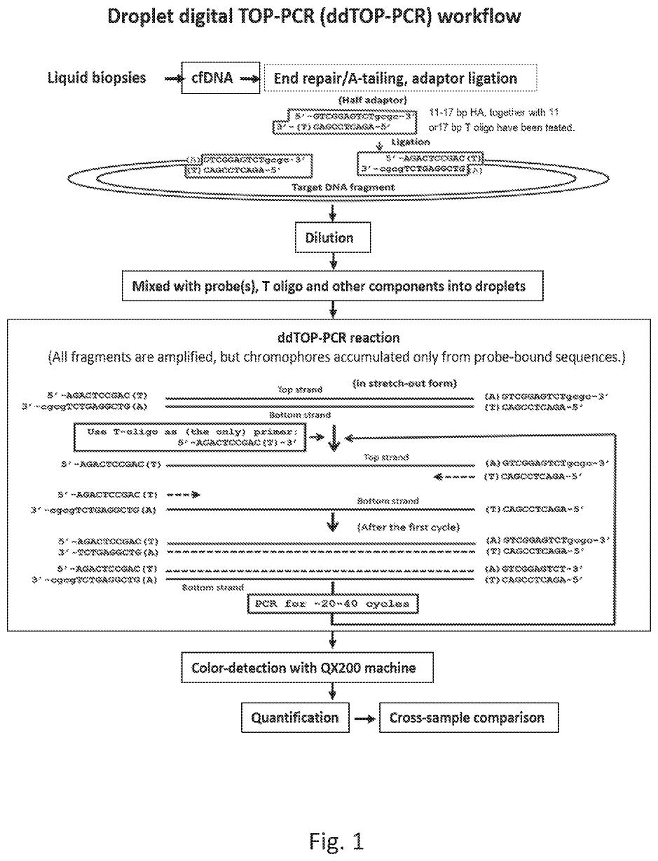

FIG. 1 is a diagram showing the procedures of the method of the present invention.

Nucleic Acid Samples

DNA samples (e.g. cfDNA samples) can be obtained from any sample containing particular nucleic acids of interest to be detected, for example, bodily fluid or tissue e.g. blood, urine, skin, hair, stool, and mucus, or an environmental sample e.g. a water sample or a food sample. The samples can be treated to isolate and purify DNAs therefrom by routine procedures such as phenol-chloroform extraction or Qiagen kit. The method of the present invention can be used for both DNA and RNA targets. For DNA samples, DNA polymerase can be used directly for amplification. For RNA samples, a reverse transcription step with reverse transcriptase will need to be first performed.

End Repair and A-Tailing

The DNA fragments in sample are end-repaired, tailed an “A” to every 3′ end, to provide 3′A-overhang DNA fragments. Conventional methods or kits are available to perform the end repair and A-tailing step such as NEBNext® Ultra End Repair/dA-Tailing Module (NEB, E7442S/L).

A Homogenous Adapter for Use in T/U Oligo Primed-P CR (TOP PCR) Amplification

A homogenous adapter is designed for use in TOP-PCR amplification. In a homogenous adapter, one strand is called T/U oligo, having an extra thymine or uracil nucleotide (T/U) at the 3′-end; and the other strand is called P oligo, having a phosphate group at the 5′-end and its 3′-end nucleotide has no extra T or U. The adapter may be a blunt-sticky (i.e., one end is blunt and the other end is sticky) or double sticky (i.e., both ends are sticky) adapter. In some embodiments, for a “blunt-sticky” adapter, the P oligo is one base shorter and is complementary to the T/U oligo except at the 3′-end T/U of the T/U oligo. In some embodiments, for a “double-sticky” adapter, the P oligo is longer than the T/U oligo. A homogenous adapter as used herein requires (i) T/U oligo has an extra 3′-T/U (i.e., ‘T’ or ‘U’ overhang at the 3′ end) and has no 5′-phosphate; (ii) P oligo needs a 5′-phosphate; and (iii) T/U oligo is complimentary to P oligo except at the 3′-T/U overhang of T/U oligo. T/U oligo and P oligo may vary in length and sequence. In some examples, P oligo sequence is 5′-GTCGGAGTCTgcgc-3′ (SEQ ID NO:24), and T/U-oligo sequence is 5′-AGACTCCGAC(T)-3′ (SEQ ID NO:23). In some examples, P oligo sequence is 5′-GT CGGAGT CTA GCG CT -3′ (SEQ ID NO: 2), and T/U-oligo sequence is 5′-AGC GCT AGA CTC CGA CT -3′ (SEQ ID NO: 1). Moreover, in some embodiments, “3′-U”, instead of “3′-T”, can be used, so that the double-stranded half adapter (HA) can be completely trimmed off after amplification by using “user enzyme” (Uracil-Specific Excision Reagent, by NEB).

Ligation of a homogenous adapter to DNA fragments

The homogenous adapter is ligated to both termini of the 3′A-overhang DNA fragments after the end repair and A-tailing step, to produce adapter-ligated DNA fragments, wherein the 3′-T/U of the T/U oligo of the adapter is complementary to the 3-′A overhang of the 3′-A overhang DNA fragments. The ligation can be conducted in a proper ligation mixture including the adapter, the 3′-A overhang DNA fragments, ligase and ligation buffer, under a proper condition e.g. about 25° C. overnight in a thermocycler. The ligation mixture can be directly subjected to PCR amplification with or without DNA purification.

PCR/Detection Reagents

After ligation, the sample is combined with PCR/detection reagents to provide amplification/detection-ready sample. PCR reagents typically include primers, nucleotides, polymerase, and buffers. Detection reagents typically include one or more detectable probe(s). These input reagents may be provided as individual reagents to be added separately into the sample, or some or all of the reagents may be provided as reagent mixes to be added into the sample in a premixed form. PCR reagents typically include a buffer which is selected to facilitate the amplification reaction. Magnesium ions e.g. MgCl₂ are usefully included in the buffer. PCR reagents also include nucleotides. The four dNTPs (dATP, dCTP, dGTP and dTTP) are typically provided in equimolar concentrations. A variety of PCR polymerases may be used in the same to perform amplification. Suitable polymerases will often have optimal activity at about 75° C. and the ability to retain that activity after prolonged incubation, e.g., at temperatures greater than 95° C. Useful polymerases may include, for example, Taq DNA polymerases, such as AmpliTaq®, AmpliTaq Gold®, the Stoffel fragment of AmpliTaq®, and others. Specifically, the PCR reagents include a single-type primer having the nucleic acid sequence of the T/U oligo strand as described herein as both forward and reverse primers for amplification. Detection reagents including a probe with specificity for a target nucleic acid can be added to the sample and a detectable signal e.g. a fluorescence signal caused by degradation of the probe can be detected.

Fractionation/Dilution

The amplification/detection-ready sample is fractioned into multiple partitions to an extent that each containing limited copies of the adapter-ligated NA fragments. Specifically, most partitions may contain no copies, others only one copy, still others could contain two copies, three copies, and even higher numbers of copies. Copies per partition may be adjusted as needed. In some embodiments, the fractionation is carried out to an extent that more than 50% of the partitions containing no more than one copy of the adapter-ligated NA fragment(s). In some embodiments, the fractionation is carried out to an extent that each partition contains at least one copy of the adapter-ligated NA fragment(s) e.g. 1-5 copies in each partition. The fractionation can be carried out in emulsion droplets or in a multiple-well as known in the art, for example, as described in Lodrini et al., 2017 and U.S. Patent Application Publication No. 2009/0053719 and 20150099644, the entire content of which is incorporated herein by reference. In some embodiments, the sample is divided into a plurality of small reactions in oil droplets through a water oil emulsion technique. The oil droplets are made using a droplet generator. Typically, approximately 20,000 oil droplets are formed from each 20 μl sample.

T/U Oligo Primed-PCR Amplification

After fractionation, the PCR is performed using a free T/U oligo as the only PCR primer for amplification. As used herein, a free T/U oligo is a single primer having the nucleic acid sequence of the T/U oligo strand as described herein. A free T/U oligo refers to a T/U oligo that is not formed in an adapter with its complementary P oligo. In this way, all the DNA fragments ligated with the adapter at both end are amplified in equal proportion.

Detection/Quantification

Detection of the target DNAs can be performed by a number of methods known in the art such as flow cytometry using fluorescent probes.

In certain embodiments, a probe having a detectable label such as a fluorophore (e.g. FAM, 6-fluorescein amidite) is used for detection. A fluorescent probe has complementary sequences which specifically hybridize with a target nucleic acid fragment where the fluorophore is released from the probe during amplification of the target nucleic acid fragment by PCR (generating a positive signal, indicating sequence detected) while the fluorophore is not released from the probe if target nucleic acid fragment is not present or no target nucleic acid fragment is amplified (generating a negative signal, indicating sequence not detected). In some embodiment, a detectable probe is present in the PCR mixture.

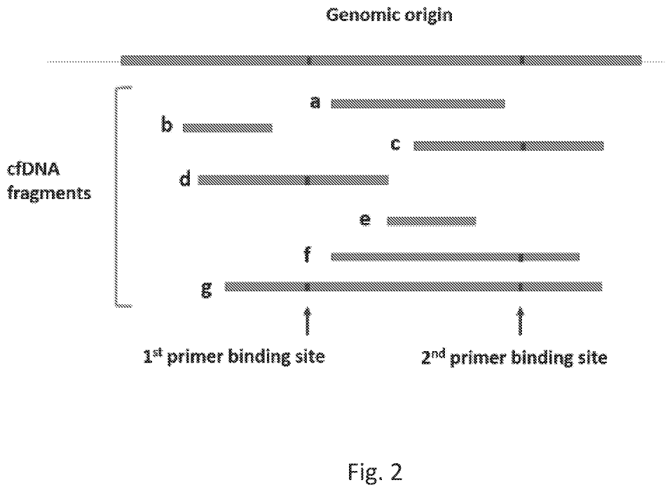

Conventional digital PCR uses probes that are typically designed based on a specific site (e.g. a mutation position) in the internal region of nucleic acids of interest which is amplified using paired primers; such probe cannot cover all genetic variations and thus restrict the testing of other potential biomarkers in the nucleic acids. In the contrast, the method of the present invention allows amplification of all nucleic acid fragments in the sample and therefore shotgun probes capable of specifically hybridizing with any region within the target nucleic acids can be used and detection of target nucleic acids can be achieved with increased sensitivity. See FIG. 2 as one particular embodiment. According to the present invention, not only fragment “g” (covering two primer binding sites) but also other fragments “a” to “f” (with only one primer binding site or even without any of the primer binding sites), all originating from the same target pathogenic nucleic acid, can be detected, for example, using shotgun probe(s) with detectable label(s) capable of specifically hybridizing with any region within the target DNAs, such that sensitivity of detection is increased and false negatives can be minimal.

After detection, the number of positive partitions (sequence detected) versus negative partitions (sequence not detected) is counted to determine the estimated amount of the target nucleic acid fragment in the sample. The quantification can be carried out according to a method known in the art, for example, as described in Lodrini et al., 2017. In certain embodiments, droplets after PCR amplification can be measured in the QX200 ddPCR Droplet Reader and target copy number is analyzed using QuantaS oft analysis software.

Also provided is a kit for performing the method for detecting nucleic acid fragment(s) in samples as described herein. In particular, the kit comprises

(i) adapter ligation reagents comprising a homogenous adapter, a ligation buffer and a ligase, wherein the homogenous adapter comprises a P oligo strand carrying a 5′-phosphate and a T/U oligo strand carrying a 3′-T or 3-U without a 5′-phosphate, wherein the T/U oligo strand is complimentary to the P oligo strand except at the 3′-T or 3′-U of the T/U oligo strand, wherein the homogenous adapter is capable of ligating to the nucleic acid fragment(s) at both ends, wherein the nucleic acid fragment(s) has a 3′-A overhang;

(ii) PCR reagents comprising a single-type primer having the nucleic acid sequence of the T/U oligo strand, dNTPs (dATP, dCTP, dGTP and dTTP), a PCR buffer, and a DNA polymerase; and

(iii) detection reagents comprising one or more detectable probes having complementary sequences specifically hybridizing with the nucleic acid fragment(s).

In some embodiment, the kit further comprises instructions for use. Specifically, the instructions for use comprise instructions for performing the method of the present invention comprising steps (a) to (g).

Utilities and Advantages of the Present Invention

The method of the present invention is useful in diagnosis or prognosis, especially in cfDNA-based detection. Detection of body fluid samples containing cfDNAs has been described a non-invasive approach useful for diagnostics of genetic defects, infectious origins and diseases, especially valuable in early detection, and also prognosis at least because cfDNA remains available even if the diseased tissues e.g. tumors are removed. However, conventional PCR including qPCR or dPCR, designed as template-dependent requiring at least one one pair of primers, is not suitable for cfDNA detection since the cfDNA as the templates are usually not in good quality and quantity such that sensitivity has limitation and bias may happen if PCR cycles increase. The method of the present invention, by using a homogenous adapter composed of a P oligo and a T/U oligo ligating to the DNAs and the T/U oligo as a single primer, is able to amplify all the DNAs in a sample with any initial quantity, in equal proportion, and detection of target DNAs with specific probes can be carried out to increase the sensitivity without substantial bias (false negative).

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

Although digital PCR (dPCR) is a powerful method for analysis of genetic mutations and copy number variations (CNVs), it is not suitable for analysis of cell-free DNA (cfDNA) in clinical samples due to the fact that cfDNA fragments are not only at low quantity but also highly fragmented, which may introduce false negatives to conventional digital PCR method that uses dual primers for amplification. To overcome the problem, we have developed droplet digital T/U oligo-primed polymerase chain reaction (ddTOP-PCR) which relies on adapter-dependent, non-discriminatory amplification of all cfDNA fragments, followed by detection using oligo probe(s) labeled with FAM or HEX that generates chromophores from specific target(s) during elongation. The fluorescent signals are subsequently detected by QX200 ddPCR machine. Results showed that ddTOP-PCR was able to detect 5′ primer binding site-deleted N-myc sequences, while ddPCR could not. Further testing with samples containing fragments of 5′ primer binding site-deleted construct and/or dual primer binding site-intact N-myc construct, ddTOP-PCR showed ˜14% increase in sensitivity, compared to conventional ddPCR, although with compromised signal intensity. These proof-of-concept experiments demonstrate the superiority of ddTOP-PCR over its counterpart for the analysis of cfDNA in liquid biopsies.

1. Material and Methods

1.1. Cloning of N-myc Sequence to be Used as Standard for Analysis

To make standard for optimization of experimental conditions and analysis of copy number variation, vectors carrying N-myc genetic sequences were constructed by using primers designed to target the N-myc sequence of human based on the Homo sapiens chromosome 2, GRCh38.p12 Primary Assembly (Accession Number: NC_000002.12) retrieve from NCBI database. N-myc amplicon (157 bp) was cloned/amplified from the genomic DNA of Be2C cell line using forward primer 5′-AAG GGG TGC TCT CCA ATT CT-3′ (SEQ ID NO: 13) and reverse primer 5′-CGG TTT AGC CAC CAA CTT TC-3′ (SEQ ID NO: 14). NAGK amplicon (172 bp) was cloned/amplified from the genomic DNA of Be2C cell line using forward primer 5′-CCC CTT TCC CGC TAT ATC TT-3′ (SEQ ID NO: 15) and reverse primer 5′-ATG CAG GGT TTG ATG GGA TA-3′ (SEQ ID NO: 16). Polymerase chain reaction (PCR) using Q5 High-Fidelity 2× Master Mix (NEB, MA, US) was carried out to amplify the target amplicon. PCR reaction was performed in a mixture (50 μl) containing the respective primer set, Be2C genomic DNA (10 ng), 1× Q5 High-Fidelity Master Mix. The mixture was then incubated at 98° C. for 1 min followed by 30 cycles of 98° C. for 20 s, 60° C. for 30 s and 72° C. for 10 s. The mixture was later incubated at 72° C. for 2 min for final elongation. The PCR products were then analysed and amplicons with the expected size were extracted from a 2% agarose gel prior gel DNA extraction using QlAquick Gel Extraction Kit (Qiagen, NW, GE) following the instruction manual.

1.2 Ligation of Half-Adapter (HA) to N-myc and NAGK Standard Templates

Half-adapter (HA) was first prepared by annealing 16-mer P oligo (5′-pGTCGGAGTCTAGCGCT-3C6-3′) (SEQ ID NO: 2) and 17-mer T/U oligo (5′-AmC6-AGCGCTAGACTCCGACT-3′) (SEQ ID NO: 1) at 1:1 molar ratio by incubating at 95° C. for 5 min followed by gradually reduction of temperature to 4° C. using a thermocycler.

Before ligation, 10 ng of 157 bp N-myc amplicon and 172 bp NAGK amplicon were first end-repaired and 3′ A-tailed using NEBNext® Ultra™ II DNA Library Prep Kit for Illumina sequencing (NEB, MA, US) with slight modification. Then, a 50:1 ratio of HA vs. amplicon, for example, was used for ligation and the reactions were incubated at 16° C. overnight in a thermocycler.

When needed, direct TOP-PCR was performed on the ligation mixture without purification. The reaction was performed in a mixture (50 μl) using T/U oligo (5′-AGCGCTAGACTCCGACT-3′ (SEQ ID NO: 1); 1 μM), ligation mixture (5 μ), 1× Phusion HF reaction buffer (Thermo Fisher Scientific, MA, US) containing Mg²⁺ (1.5 mM), Phusion High Fidelity Hot Start DNA polymerase (1 U) and dNTPs (1 mM). The mixture was then incubated at 98° C. for 1 min preceded 30 cycles of 98° C. for 20 sec, 57° C. for 30 sec and 72° C. for 1 min for sheared gDNA while 10 sec for the standard templates. The mixture was later incubated at 72° C. for 5 min for final elongation. The TOP-PCR amplified N-myc and NAGK namely HA-N-myc-HA and HA-NAGK-HA respectively were purified using QIAquick PCR Purification Kit (Qiagen, NW, GE) following the instruction manual and quantified using the Qubit dsDNA HS Assay Kit (Thermo Fisher Scientific, MA, US).

1.3 Construction of Standard Template

In order to test the T/U oligo and probes specificity. Different ratio combination of pHE-N-myc-HA and pHE-NAGK-HA templates were prepared. A ratio ranging from 100:1 to 1:100 of pHE-N-myc-HA and pHE-NAGK-HA templates in a 10-fold dilution order were prepared. The ddPCR mixture were prepared with slight modification. The ddPCR reaction (20 μl) consists T/U oligo primer (8 μM), Mpb1+2 (0.25 μM), Npb2 (0.25 μM), DNA template (4.0 μl), 1× ddPCR™ Supermix for Probes (No dUTP) (Bio-Rad, CA, US) prior droplet generation. The ddPCR droplet mixtures were prepared and ddPCR reaction were performed as aforementioned.

1.4 Testing of gDNA-HA

Specific amplification of N-myc and NAGK targets in gDNA mixture were performed using ddTOP-PCR. The gDNA-HA template constructed were used for this experiment using the aforementioned ddPCR parameter. Different input amount of sheared gDNA-HA ranging from 100 ng to 100 pg in a 10-fold dilution order were used for this experiment. The ddPCR reaction (20 μl) consists T/U oligo primer (8 μM), Mpb1+2 (0.25 μM), Npb2 (0.25 μM), gDNA-HA template (100 ng to 100 p g), 1× ddPCR™ Supermix for Probes (No dUTP) (Bio-Rad, CA, US) prior droplet generation. The ddPCR droplet mixtures were prepared and ddPCR reaction were performed as aforementioned.

1.5 Construction of Vectors Carrying HA-N myc-HA and HA-NAGK-HA Sequence to be Used as Standard forAnalysis

The HA-N myc-HA and HA-NAGK-HA constructs was then cloned using HE Swift Cloning Kit (Toolbiotech, TW), followed by transformation into DH5a competent cells and plating onto ampicillin LB agar plate. Bacteria colonies were screened and sequenced via Sanger sequencing to verify the sequences after plasmid extraction using QlAprep Spin Miniprep Kit (Qiagen, NW, GE).

The standard templates for ddTOP-PCR were produced by amplifying the recombinant plasmid harbouring the correct HA-N myc-HA and HA-NAGK-HA sequences in a 100 μl PCR reaction containing the pHE-F primer (5′-CGA CTC ACT ATA GGG AGA GCG GC-3′; SEQ ID NO: 17, 0.5 μM), pHE-R primer (5′-AA GAA CAT CGA TTT TCC ATG GCA G-3′; SEQ ID NO: 18, 0.5 μM), DNA (1 ng), 1× QS High-Fidelity Master Mix. The mixture was then incubated at 98° C. for 1 min followed by 30 cycles of 98° C. for 20 s, 64° C. for 30 s and 72° C. for 10 sec. The mixture was later incubated at 72° C. for 2 min for final elongation. The PCR amplicon namely pHE-HA-N myc-HA and pHE-HA-NAGK-HA with size of 309 bp and 325 bp respectively were purified using QIAquick PCR Purification Kit (Qiagen, NW, GE) and quantified.

1.6 Construction 5′-Deleted N-myc and NAGK Constructs to be Used as Standard for Analysis

The 5′ primer binding site-deleted construct of N-myc was amplifying with forward primer (5′-AGC GCT AGA CTC CGA CTT CAC TAA AGT TCC TTC CAC CCT CTC CTG GGG AG-3′) (SEQ ID NO: 19) and reverse primer (5′ -AGC GCT AGA CTC CGA CTT AGC CAC CAA CTT TCT CCA ATT TTA TTC CTC AG-3′) (SEQ ID NO: 20) by Q5 High-Fidelity Master Mix. The 5′ primer binding site-deleted construct of NAGK was amplifying with forward primer (5′-AGC GCT AGA CTC CGA CTG TGT TGC CCG AGA TTG ACC CGG TGA GTT GAG GT-3′) (SEQ ID NO: 21) and reverse primer (5′-AGC GCT AGA CTC CGA CTA TGC AGG GTT TGA TGG GAT AGT CCC ATC-3′) (SEQ ID NO: 22) by Q5 High-Fidelity Master Mix. The PCR amplicon namely HA-N myc-F del-HA and HA-NAGK-F del-HA with size of 160 bp and 146 bp respectively were purified using QlAquick PCR Purification Kit (Qiagen, NW, GE) and quantified.

1.7 PCR Primers Used for Amplification

Paired primer sequences for the ddPCR were retrieved from Lodrini et al., with slight modification on the sequence (Lodrini et al., 2017). The primers and probes were synthesized by Integrated DNA Technology (IDT). Practically, for the amplification of N-myc sequence, forward primer (5′-GTG CTC TCC AAT TCT CGC CT-3′) (SEQ ID NO: 3) and reverse primer (5′-GAT GGC CTA GAG GAG GGC T-3′) (SEQ ID NO: 4) are employed.

1.8 Probes for Detection

For detecting N-myc amplifications, 3 probes were designed (as shown below). These include 1) probe Mpbl (FAM-N-myc probe): /56-FAM/CAC TAA AGT/ZEN/TCC TTC CAC CCT CTC CT/3IABkFQ/(SEQ ID NO: 10); 2) probe Mpbl+1 (FAM-N-myc Probe +lnt): /56-FAM/CAC TAA AGT/ZEN/TCC TTC CAC CCT CTC CTG/3IABkFQ/(SEQ ID NO: 11); and 3) probe Mpb1+2 (FAM-N-myc Probe +2nt): /56-FAM/CAC TAA AGT/ZEN/TCC TTC CAC CCT CTC CTG G/3IABkFQ/(SEQ ID NO: 12). This initial testing used Probe Mpb1+1.

1.9 Conditions of ddPCR and ddTOP-PCR

To achieve a workable state for ddTOP-PCR and eventually to optimize its sensitivity and specificity, the lengths and experimental conditions of both PCR primers and fluorescent probes have to be tested. Moreover, conventional ddPCR has to be run side-by-side as a positive control for initial setup and thus the optimization and experimental conditions was applied to both ddTOP-PCR and ddPCR.

Based on the preliminary results we determined to use a primer concentration of 0.9 μM for gene specific primers (ddPCR control) and 32 μM for T/U oligo (ddTOP-PCR). A total of 20 μl PCR reaction comprises primer(s) (paired primers for ddPCR control or a T/U oligo primer for ddTOP-PCR), 0.25 μM probe, DNA template (2.0 μl), 1× ddPCR Supermix for probes (No dUTP) (Bio-Rad). Droplets were generated by mixing the prepared PCR reaction mixture (20 μl) with 70 μl of droplet digital PCR oils (Bio-Rad). A total of 40 μl ddPCR droplet mixture was transferred to 96-well plate and sealed prior PCR reaction with Bio-Rad T-100 thermocycler (Bio-Rad). Both ddPCR and ddTOP-PCR preparations were then placed in Bio-Rad PCR machine for amplification and chromophore generation under conditions: 95° C. for 10 min, followed by 40 cycles of 94° C. for 30 sec and 58° C. for 60 sec. Reactions were terminated by incubated at 98° C. for 10 min. After PCR reaction, the quantification of positive droplet was performed using the QX200 ddPCR Droplet Reader and analyzed using QuantaSoft analysis software (version 1.7.4, Bio-Rad).

2. Results

2.1 Detection of N-myc Gene Sequences Using ddPCR and ddTOP-PCR for Comparison

The initial testing was conducted to prove the concept that by replacing conventional PCR with TOP-PCR, one should be able to increase the sensitivity, which is presented as an increase in number of positive counts in QX200 ddPCR machine.

Samples containing only NAGK or N-myc sequence provides an easily verifiable system to improve conditions, under which experimental conditions (e.g., preparation of samples, concentrations of PCR ingredients and reaction conditions) can be adjusted based on experimental outcome.

For the testing, we prepared two types of N-myc sequence-containing fragments: one with both primer-binding sites, while the other with the 5′ primer binding site deleted, leaving only the 3′-binding site intact, together with NAGK control which also has 5′ primer binding site deleted (Table 1).

TABLE 1
Experimental design

| Template ID and sequence | ddPCR primers | ddTOP-PCR primer |
|---|---|---|
| Full N-myc construct: HA-pHE-N myc-HA (190 | N-myc forward: GTG | T oligo: 5′-AGC GCT |
| bp, SEQ ID NO: 7): | CTC TCC AAT TCT | AGA CTC CGA CT-3′ |
| | CGC CT (same as the | (SEQ ID NO: 1) |
| AGC GCT AGA CTC CGA CT AAG GGG TGC | bolded bases in the | (P oligo, 5′-GT CGG AGT |
| TCT CCA ATT CTC GCC TTC ACT AAA GTT | left box) | CTA GCG CT-3′, was |
| CCT TCC ACC CTC TCC TGG GGA GCC CTC | (SEQ ID NO: 3) | used for making half |
| CTC TAG GCC ATC ACG GGC CCT CAC CCG | N-myc reverse: GAT | adapter). |
| GTC CCC CAC CTC TCT TTT GCA GCG CAG | GGC CTA GAG GAG | (SEQ ID NO: 2) |
| TCT GAG GAA TAA AAT TGG AGA AAG TTG | GGC T (′reverse | Note: both T and P oligos |
| GTG GCT AAA CCG AGT CGG AGT CTA GCG | complementary′ to the | are italicized in the |
| CT | second bold bases in | template sequences shown |
| N-myc sequence (157 bp) (underlined) | left box) | in the first column. |
| Probe corresponding region (double-underlined) | (SEQ ID NO: 4) | - |
| 5′-deleted N-myc construct: HA-N myc-F del-HA | N-myc forward: GTG | |
| (160 bp): SEQ ID NO: 8 | CTC TCC AAT TCT | |
| AGC GCT AGA CTC CGA CT TCA CTA AAG | CGC CT (no longer | |
| TTC CTT CCA CCC TCT CCT GGG GAG CCC | present in the | |
| TCC TCT AGG CCA TCA CGG GCC CTC ACC | left box) | |
| CGG TCC CCC ACC TCT CTT TTG CAG CGC | (SEQ ID NO: 3) | |
| AGT CTG AGG AAT AAA ATT GGA GAA AGT | N-myc reverse: GAT | |
| TGG TGG CTA AGT CGG AGT CTA GCG CT | GGC CTA GAG GAG | |
| N myc-F del sequence (127 bp) (underlined) | GGC T (′reverse | |
| Probe corresponding region (double-underlined) | complementary′ to | |
| | bold bases in | |
| | left box) | |
| | (SEQ ID NO: 4) | |
| 5′-deleted NAGK construct: HA-NAGK-F del-HA | NAGK forward: TGG | |
| (146 bp): SEQ ID NO: 9 | GCA GAC ACA TCG | |
| AGC GCT AGA CTC CGA CT GTG TTG CCC | TAG CA (no longer | |
| GAG ATT GAC CCG GTG AGT TGA GGT GGG | present in the | |
| AGT GAA GGT GGG GAG CTG CTG GGT GAG | left box) | |
| GAG TGG TCC TTT CCC ACT GTG GAT GGG | (SEQ ID NO: 5) | |
| ACT ATC CCA TCA AAC CCT GCA T AGT | NAGK reverse: CAC | |
| CGG AGT CTA GCG CT | CTT CAC TCC CAC | |
| NAGK-F del sequence (113 bp) (underlined) | CTC PAC (′reverse | |
| | complementary′ to | |
| | bold bases in left | |
| | box) (SEQ ID NO: 6) | |

Highlighted regions are the genic sequences of N-myc (underlined) or NAGK (underliend), flanked by T oligo (left) and P oligo (right). All experiments used the same probe: /56-FAM/CAC TAA AGT/ZEN/TCC TTC CAC CCT CTC CTG/3IABkFQ/, which is named as Mpb1 + 1 (SEQ ID NO: 11).

We used QX200 ddPCR machine on different protocols, either ddPCR or ddTOP-PCR, to demonstrate the potential of ddTOP-PCR in detecting fragments with defective primer binding sites, which are likely to present in the pool of cfNA sample.

For the proof-of-concept testing, we prepared samples containing variable percentages of above-mentioned 5′ primer binding site-deleted templates. The initial input in each sample was calculated to be ˜12,000 copies total, and all experiments were performed by QX200 ddPCR machine using settings reported by Lodrini et al but with minor modifications (Lodrini et al., 2017). The same N-myc oligo probe sequence used by Lodrini et al was also used in this experiment.

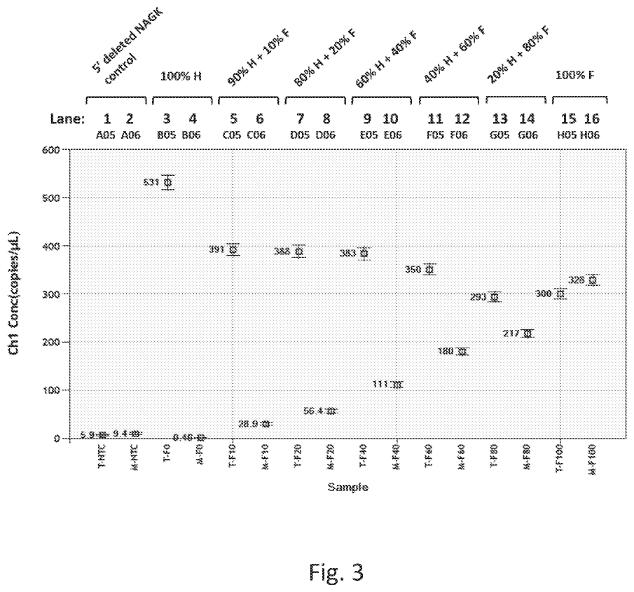

Results showed that, in general, ddTOP-PCR method, which used T oligo as the only primer for amplification, has higher counts than ddPCR, which used dual internal primers (FIG. 3).

To compare the sensitivity of ddTOP-PCR against ddPCR, we estimated the copy number inputs in the original samples (using Qubit) and the corresponding detected copy numbers and then calculate the percentages detected (Table 2).

TABLE 2
Comparison of sensitivities between ddPCR and ddTOP-PCR.

| | | Full | Detected | | (Half + | | |
|---|---|---|---|---|---|---|---|
| | | input | by | | Full) input | Detected by | |
| ID | Full % | copy no. | ddPCR | Sensitivity | copy # | ddTOP-PCR | Sensitivity |
| 1 | 0% | — | — | — | 12,000 | 10,620 | 88.5% |
| 2 | 10% | 1,200 | 578 | 48.2% | 12,000 | 7,820 | 65.2% |
| 3 | 20% | 2,400 | 1,128 | 47.0% | 12,000 | 7,760 | 64.7% |
| 4 | 40% | 4,800 | 2,220 | 46.3% | 12,000 | 7,660 | 63.8% |
| 5 | 60% | 7,200 | 3,600 | 50.0% | 12,000 | 7,000 | 58.3% |
| 6 | 80% | 9,600 | 4,340 | 45.2% | 12,000 | 5,860 | 48.8% |
| 7 | 100% | 12,000 | 6,560 | 54.7% | 12,000 | 6,000 | 50.0% |

| | | | |
|---|---|---|---|
| Average if 1 not included | 48.6% | Average if 1 not included | 58.5% |

| | |
|---|---|
| Average if 1 included | 62.8% |

Results indicate that ddPCR was able to detect about 48.6% of the templates, while ddTOP-PCR was able to detect about 58.5%-62.8% of the templates, indicating a 10%-15% increase in sensitivity from ddPCR to ddTOP-PCR. Notice that, although accuracy is influenced by bias/variations resulted from quantification device (e.g., Qubit), personal skill, QX200 machine itself, and others, the general trend for each method possesses certain degree of reliability.

Calculate Standard Deviation

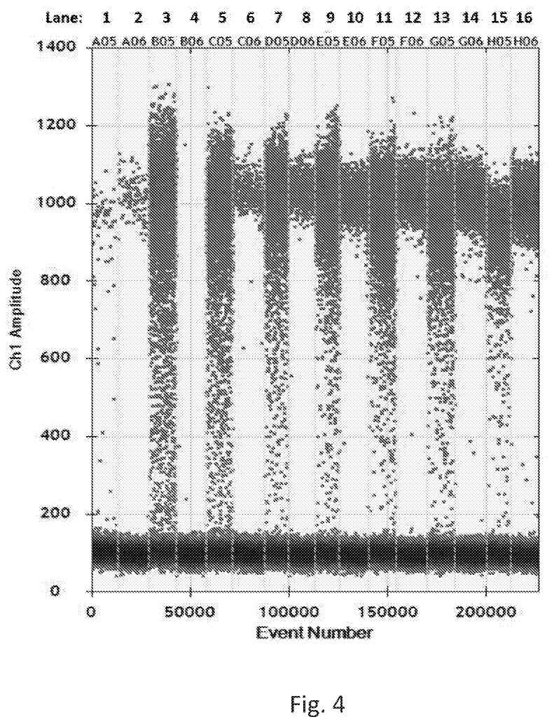

However, higher sensitivity of ddTOP-PCR is compromised with more scattered signal intensity (FIG. 4). Most of the positive signals in ddTOP-PCR droplets have lower intensity than that of ddPCR droplets. Presumably due to the fact that the usage of dual internal primers resulted in shorter and well-defined range of amplification to allow ddPCR to generate signals of higher degree of uniformity than ddTOP-PCR whose amplification initiates from the flanking adapters farther away from the dual priming sites.

As shown in all mixed samples of FIG. 4, color intensities of droplets produced from ddTOP-PCR can be either higher or low and are more scattered than that produced from ddPCR. As shown in lane 4, ddPCR was unable to detect 5′ primer binding site-deleted fragment, while ddTOP-PCR detects it with high efficiency. The first two lanes in the figure indicate low levels of false positives for NAGK templates.

Previous observations indicate that null background has clean count (0) for both ddTOP-PCR and ddPCR method (data not shown).

As demonstrated by ddPCR, amplification by internal dual primers produce more uniformed results, while on the other hand, amplification by adapter-based ddTOP-PCR has better sensitivity but compromised by lower intensity.

These data also indicate that further optimization is required. Moreover, shorter fragments seem to have advantage over longer ones.

3. Conclusions

This proof-of-concept study presents a preliminary data to demonstrate the development of ddTOP-PCR and empirically shows the feasibility of using ddTOP-PCR to improve the accuracy for cfDNA-based analysis of CNVs, genetic mutations, as well as alterations in gene expression and SNPs for diseases.

Comparing to conventional ddPCR, ddTOP-PCR has a number of advantages: 1) Digital PCR is not suitable for cfDNA analysis, because, as a template-dependent method, regular PCR requires both primer-binding sites to co-present in the same fragment. On the other hand, as an adapter-dependent PCR method, ddTOP-PCR does not have such constrain and thus is able to detect partial fragments; 2) Prior to ddTOP-PCR experiment, TOP-PCR alone can be employed to preserve low-quantity samples, while conventional PCR cannot. Since the quantity of a cfDNA sample can be extremely low, but a good method has to be able to recruit all cfDNA fragments in the analysis; as such the unbiased preservation of minute DNA fragments is crucially important; 3) ddTOP-PCR may be suitable for early detection of cancer and possibly other diseases as well, while conventional ddPCR cannot.

Cell-free DNAs fragments are severely diluted by nonspecific DNA fragments originated from normal or non-diseased cells. The conventional design of having a single DNA fragment per droplet is cost-ineffective and thus impractical. Instead, multiple copies per droplet is more suitable for cfDNA. We started with ˜4 copies of DNA fragments per droplet for the initial testing of ddTOP-PCR in QX200 machine.

Present results also indicate that further improvements in quantification devices and protocol will be helpful.

### REFERENCES

Chiu, K. P., and Yu, A. L. (2019). Application of cell-free DNA sequencing in characterization of bloodborne microbes and the study of microbe-disease interactions. PeerJ 7, e7426.

Dong, L., Meng, Y., Sui, Z., Wang, J., Wu, L., and Fu, B. (2015). Comparison of four digital PCR platforms for accurate quantification of DNA copy number of a certified plasmid DNA reference material. Sci Rep 5, 13174.

Hindson, B. J., Ness, K. D., Masquelier, D. A., Belgrader, P., Heredia, N. J., Makarewicz, A. J., Bright, I. J., Lucero, M. Y., Hiddessen, A. L., Legler, T. C., et al. (2011). High-throughput droplet digital PCR system for absolute quantitation of DNA copy number. Anal Chem 83, 8604-8610.

Lodrini, M., Sprussel, A., Astrahantseff, K., Tiburtius, D., Konschak, R., Lode, H. N., Fischer, M., Keilholz, U., Eggert, A., and Deubzer, H. E. (2017). Using droplet digital PCR to analyze MYCN and ALK copy number in plasma from patients with neuroblastoma. Oncotarget 8, 85234-85251.

Morley, A. A. (2014). Digital PCR: A brief history. Biomol Detect Quantif 1, 1-2.

Nai, Y. S., Chen, T. H., Huang, Y. F., Midha, M. K., Shiau, H. C., Shen, C. Y., Chen, C. J., Yu, A. L., and Chiu, K. P. (2017). T Oligo-Primed Polymerase Chain Reaction (TOP-PCR): A Robust Method for the Amplification of Minute DNA Fragments in Body Fluids. Sci Rep 7, 40767.

Taylor, S. C., Laperriere, G., and Germain, H. (2017). Droplet Digital PCR versus qPCR for gene expression analysis with low abundant targets: from variable nonsense to publication quality data. Sci Rep 7, 2409.

Vogelstein, B., and Kinzler, K. W. (1999). Digital PCR. Proc Natl Acad Sci U S A 96, 9236-9241.

Wagner, J. (2012). Free DNA--new potential analyte in clinical laboratory diagnostics? Biochem Med (Zagreb) 22, 24-38.

Zhao, Y, Xia, Q., Yin, Y, and Wang, Z. (2016). Comparison of Droplet Digital PCR and Quantitative PCR Assays for Quantitative Detection of Xanthomonas citri Subsp. citri. PLoS One 11, e0159004.

What is claimed is:

1. A method for analyzing nucleic acids in a sample, said sample containing one or more linear, double stranded nucleic acid fragment(s) (NA fragment(s)), said method comprising the steps of:
(a) subjecting the sample to a 3′-A tailing reaction allowing for adding an adenine nucleotide (A) to the 3′-tail of the NA fragment(s) to produce 3′-adenine nucleotide (3′-A) overhang nucleic acid fragment(s) (3′-A overhang NA fragment(s));
(b) providing a 3′-thymine (T) or 3′-uracil (U) nucleotide overhang double-stranded homogenous adapter which comprises a P oligo strand carrying a 5′-phosphate for ligation to the 3′-A overhang NA fragment(s) and a T/U oligo strand carrying a 3′-T or 3′-U without a 5′-phosphate, wherein the T/U oligo strand is complimentary to the P oligo strand except at the 3′-T or 3′-U of the T/U oligo strand;
(c) subjecting the sample of (a) to a ligation reaction allowing for ligating the homogenous adapter to the 3′-A overhang NA fragment(s) at both ends to produce adapter-ligated nucleic acid fragment(s) (adapter-ligated NA fragment(s));
(d) combining the sample of (c) with polymerase chain reaction (PCR) reagents and detection reagents to provide amplification/detection-ready sample, wherein the PCR reagents include a single-type primer having the nucleic acid sequence of the T/U oligo strand for amplification and the detection reagents include one or more fluorescent probe(s) producing fluorescent signals and specifically hybridizing with the NA fragment(s) for detection;
(e) partitioning the amplification/detection-ready sample of (d) into a plurality of partitions, each containing limited copy of the adapter-ligated NA fragment(s);
(f) performing PCR in each partition using the adapter-ligated NA fragment(s) as template(s) and the single-type primer as both forward and reverse primers to amplify the adapter-ligated NA fragment(s); and
(g) assessing the fluorescent signal(s) in each fraction.

2. The method of claim 1, wherein step (g) includes detecting the fluorescent color (signal) for all partitions containing amplified adapter-ligated NA fragment(s) and counting the number of factions with expected fluorescent color and thus determining the estimated amount of the NA fragment(s) in the sample.

3. The method of claim 1, wherein in step (e), more than 50% of the partitions containing no more than one copy of the adapter-ligated NA fragment(s).

4. The method of claim 1, wherein in step (e), each partition contains at least one copy of the adapter-ligated NA fragment(s).

5. The method of claim 1, wherein the NA fragment(s) comprises a nucleic acid sequence indicative of a healthy/diseased state of the subject.

6. The method of claim 1, wherein the homogenous adapter of step (b) does not self-ligate.

7. The method of claim 1, wherein the homogenous adapter of step (b) has 3′-T or 3′-U overhang in the T/U oligo strand and 3′-non-A overhang in the P oligo strand.

8. The method of claim 1, wherein the homogenous adapter of step (b) has one end which is 3′-T overhang and the other end which is a blunt end.

9. The method of claim 1, wherein the sample is obtained from a body fluid sample.

10. The method of claim 1, wherein the NA fragment(s) in the sample are cell-free DNAs.

11. The method of claim 1, wherein prior to step (a) further comprising performing an end-repair reaction to the NA fragment(s).

12. The method of claim 1, wherein the PCR of step (0 is performed by oil emulsion or droplet PCR, or well-based PCR.

13. The method of claim 1, wherein the assaying of step (g) is performed by flow cytometry using fluorescent probes.

14. The method of claim 1, further comprising subjecting the sample prior to step (a) to a reverse transcription-PCR (RT-PCR) to convert the RNAs to linear, double-stranded complementary DNA (cDNA).

15. A kit for performing a method for measuring the quantity of nucleic acid fragment(s) in a sample, comprising
(i) adapter ligation reagents comprising a homogenous adapter, a ligation buffer and a ligase, wherein the adapter comprises a P oligo strand carrying a 5′-phosphate and a T/U oligo strand carrying a 3′-T or 3-U without a 5′-phosphate, wherein the T/U oligo strand is complimentary to the P oligo strand except at the 3′-T or 3′-U of the T/U oligo strand, wherein the homogenous adapter is capable of ligating to the nucleic acid fragment(s) at both ends, wherein the nucleic acid fragment(s) has a 3′-A overhang;
(ii) PCR reagents comprising a single-type primer as the sole primer having the nucleic acid sequence of the T/U oligo strand, dNTPs, a PCR buffer, and a DNA polymerase; and
(iii) detection reagents comprising one or more detectable probes having complementary sequences specifically hybridizing with the nucleic acid fragment(s).

16. The kit of claim 15, further comprises instructions for use, wherein the instructions for use comprise instructions for performing a method comprising the steps of:
(a) subjecting the sample to a 3′-A tailing reaction allowing for adding an adenine nucleotide (A) to the 3′-tail of the NA fragment(s) to produce 3′-adenine nucleotide (3′-A) overhang nucleic acid fragment(s) (3′-A overhang NA fragment(s));
(b) providing a 3′-thymine (T) or 3′-uracil (U) nucleotide overhang double-stranded homogenous adapter which comprises a P oligo strand carrying a 5′-phosphate for ligation to the 3′-A overhang NA fragment(s) and a T/U oligo strand carrying a 3′-T or 3′-U without a 5′-phosphate, wherein the T/U oligo strand is complimentary to the P oligo strand except at the 3′-T or 3′-U of the T/U oligo strand;
(c) subjecting the sample of (a) to a ligation reaction allowing for ligating the homogenous adapter to the 3′-A overhang NA fragment(s) at both ends to produce adapter-ligated nucleic acid fragment(s) (adapter-ligated NA fragment(s));
(d) combining the sample of (c) with polymerase chain reaction (PCR) reagents and detection reagents to provide amplification/detection-ready sample, wherein the PCR reagents include a single-type primer having the nucleic acid sequence of the T/U oligo strand for amplification and the detection reagents include one or more fluorescent probe(s) producing fluorescent signals and specifically hybridizing with the NA fragment(s) for detection;
(e) partitioning the amplification/detection-ready sample of (d) into a plurality of partitions, each containing limited copy of the adapter-ligated NA fragment(s);
(f) performing PCR in each partition using the adapter-ligated NA fragment(s) as template(s) and the single-type primer as both forward and reverse primers to amplify the adapter-ligated NA fragment(s); and
(g) assessing the fluorescent signal(s) in each fraction.


SEQUENCE LISTING (24 sequences)

SEQ ID NO: 1 - DNA, 17 bp, artificial sequence
  T oligo (17 mer)
agcgctagactccgact

SEQ ID NO: 2 - DNA, 16 bp, Artificial Sequence
  P oligo (16 mer)
gtcggagtctagcgct

SEQ ID NO: 3 - DNA, 20 bp, Artificial Sequence
  N-myc forward (ddPCR primer)
gtgctctccaattctcgcct

SEQ ID NO: 4 - DNA, 19 bp, Artificial Sequence
  N-myc reverse (ddPCR primer)
gatggcctagaggagggct

SEQ ID NO: 5 - DNA, 20 bp, Artificial Sequence
  NAGK forward (ddPCR primer)
tgggcagacacatcgtagca

SEQ ID NO: 6 - DNA, 21 bp, Artificial Sequence
  NAGK reverse (ddPCR proimer)
caccttcactcccacctcaac

SEQ ID NO: 7 - DNA, 190 bp, Artificial Sequence
  HA-N myc-HA
agcgctagactccgactaaggggtgctctccaattctcgccttcactaaagttccttccaccctctcctggggagccctcctctaggccatcacgggccctcacccggtcccccacctctcttttgcagcgcagtctgaggaataaaattggagaaagttggtggctaaaccgagtcggagtctagcgct

SEQ ID NO: 8 - DNA, 160 bp, Artificial Sequence
  HA-N myc-F del-HA
agcgctagactccgacttcactaaagttccttccaccctctcctggggagccctcctctaggccatcacgggccctcacccggtcccccacctctcttttgcagcgcagtctgaggaataaaattggagaaagttggtggctaagtcggagtctagcgct

SEQ ID NO: 9 - DNA, 146 bp, Artificial Sequence
  HA-NAGK-F del-HA
agcgctagactccgactgtgttgcccgagattgacccggtgagttgaggtgggagtgaaggtggggagctgctgggtgaggagtggtcctttcccactgtggatgggactatcccatcaaaccctgcatagtcggagtctagcgct

SEQ ID NO: 10 - DNA, 26 bp, Artificial Sequence
  probe Mpb1
cactaaagttccttccaccctctcct

SEQ ID NO: 11 - DNA, 27 bp, Artificial Sequence
  probe Mpb1+1
cactaaagttccttccaccctctcctg

SEQ ID NO: 12 - DNA, 28 bp, Artificial Sequence
  probe Mpb1+2
cactaaagttccttccaccctctcctgg

SEQ ID NO: 13 - DNA, 20 bp, Artificial Sequence
  N myc forward (amplicon primer)
aaggggtgctctccaattct

SEQ ID NO: 14 - DNA, 20 bp, Artificial Sequence
  N myc reverse (amplicon primer)
cggtttagccaccaactttc

SEQ ID NO: 15 - DNA, 20 bp, Artificial Sequence
  NAGK forward (amplicon primer)
cccctttcccgctatatctt

SEQ ID NO: 16 - DNA, 20 bp, Artificial Sequence
  NAGK reverse (complicon primer)
atgcagggtttgatgggata

SEQ ID NO: 17 - DNA, 23 bp, Artificial Sequence
  pHE-F primer
cgactcactatagggagagcggc

SEQ ID NO: 18 - DNA, 24 bp, Artificial Sequence
  pHE-R primer
aagaacatcgattttccatggcag

SEQ ID NO: 19 - DNA, 50 bp, Artificial Sequence
  N myc forward primer (5′del)
agcgctagactccgacttcactaaagttccttccaccctctcctggggag

SEQ ID NO: 20 - DNA, 50 bp, Artificial Sequence
  N myc reverse (5′del)
agcgctagactccgacttagccaccaactttctccaattttattcctcag

SEQ ID NO: 21 - DNA, 50 bp, Artificial Sequence
  NAGK forward primer (5′del)
agcgctagactccgactgtgttgcccgagattgacccggtgagttgaggt

SEQ ID NO: 22 - DNA, 45 bp, Artificial Sequence
  NAGK referse primer (5′del)
agcgctagactccgactatgcagggtttgatgggatagtcccatc

SEQ ID NO: 23 - DNA, 11 bp, Artificial Sequence
  T oligo (11 mer)
agactccgact

SEQ ID NO: 24 - DNA, 14 bp, Artificial Sequence
  P oligo(14 mer)
gtcggagtctgcgc